(19) United States
(12) Patent Application Publication
(10) Pub. No.: US 2025/0320244 A1
(43) Pub. Date: Oct. 16, 2025

(54) Click-Labeled Nucleosides and Phosphoramidites

(71) Applicant: SomaLogic Operating Co., Inc., Boulder, CO (US)

(72) Inventors: Catherine R. Fowler, Boulder, CO (US); Brian E. Ream, Boulder, CO (US); John C. Rohloff, Boulder, CO (US)

(73) Assignee: SomaLogic Operating Co., Inc., Boulder, CO (US)

(21) Appl. No.: 19/108,488

(22) PCT Filed: Sep. 15, 2023
(86) PCT No.: PCT/US2023/032859

§ 371 Date: Mar. 4, 2025

Related U.S. Application Data
(60) Provisional application No. 63/407,282, filed on Sep. 16, 2022.

(51) Int. Cl. C07H 19/073; C07H 1/00; C07H 21/04

(52) U.S. Cl. CPC C07H 19/073; C07H 1/00; C07H 21/04

(57) ABSTRACT

Click-labeled uridine bases, nucleosides, and phosphoramidites are provided, including improved methods of synthesis, oligonucleotides comprising the click-labeled nucleosides, methods of synthesizing spin-labeled oligonucleotides using click-labeled nucleotides, and spin-labeled oligonucleotides comprising click-labeled nucleosides.

### CROSS-REFERENCE TO RELATED APPLICATIONS

This application claims the benefit of priority of U.S. Provisional Application No. 63/407,282, filed Sep. 16, 2022, which is incorporated by reference herein in its entirety for any purpose.

### FIELD

This disclosure relates to the field of click-labeled uridine bases, nucleosides, and phosphoramidites, including improved methods of synthesis, oligonucleotides comprising the click-labeled nucleosides, methods of synthesizing spin-labeled oligonucleotides using click-labeled nucleotides, and spin-labeled oligonucleotides comprising click-labeled nucleosides.

### BACKGROUND

Quantum sensing based on nitrogen vacancy (NV) centers in diamond has emerged as a powerful technology that enables the detection of individual proteins and DNA molecules. A NV center can detect binding through a shift in the transition frequency of a spin-labeled oligonucleotide. Further, the detection of a binding event at a single-molecule level via an electron paramagnetic resonance measurement (EPR) signature would remove the ambiguity associated with non-specific adsorption in existing fluorescent techniques.

There remains a need in the art for alternative click-labeled bases, nucleosides, oligonucleotides, and phosphoramidites to enable quantum sensing of, for example, binding events.

### SUMMARY

In some embodiments, a compound having the structure

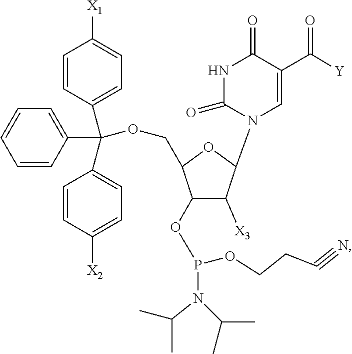

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy. In some embodiments, Y is

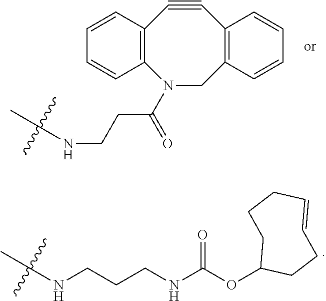 [SMILES: *NCCC(=O)N1Cc2ccccc2C#Cc2ccccc21 ; *NCCCNC(=O)OC1CC/C=C/CCC1]

In some embodiments, X₁ and X₂ are both methoxy. In some embodiments, X₃ is hydrogen. In some embodiments, X₃ is methoxy. In some embodiments, X₃ is fluoro. In some embodiments, X₃ is tert-butyldimethylsilyloxy. In some embodiments, Y is

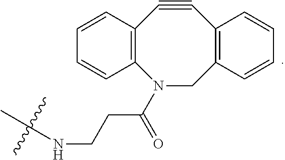 [SMILES: *NCCC(=O)N1Cc2ccccc2C#Cc2ccccc21]

In some embodiments, Y is

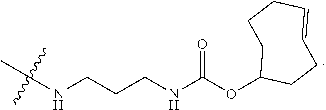 [SMILES: *NCCCNC(=O)OC1CC/C=C/CCC1]

In some embodiments, a compound provided herein is selected from:

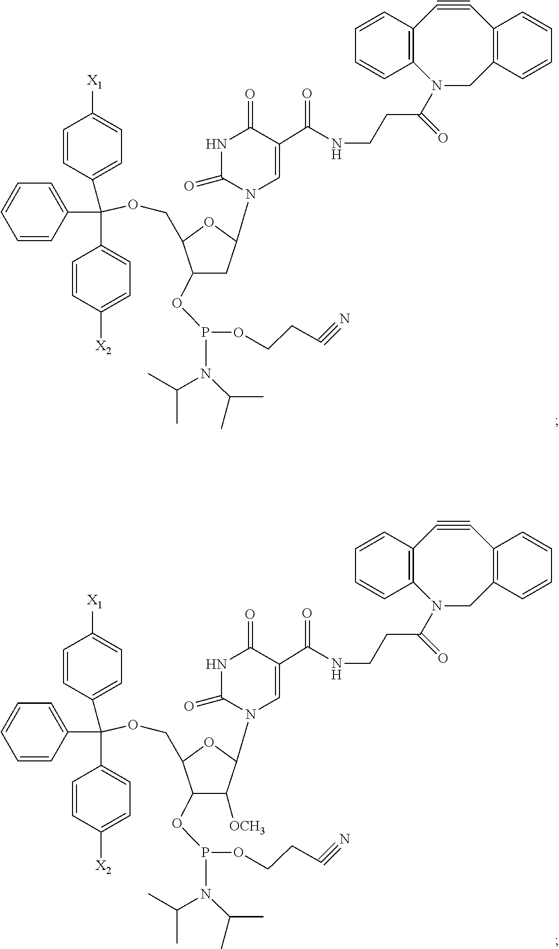

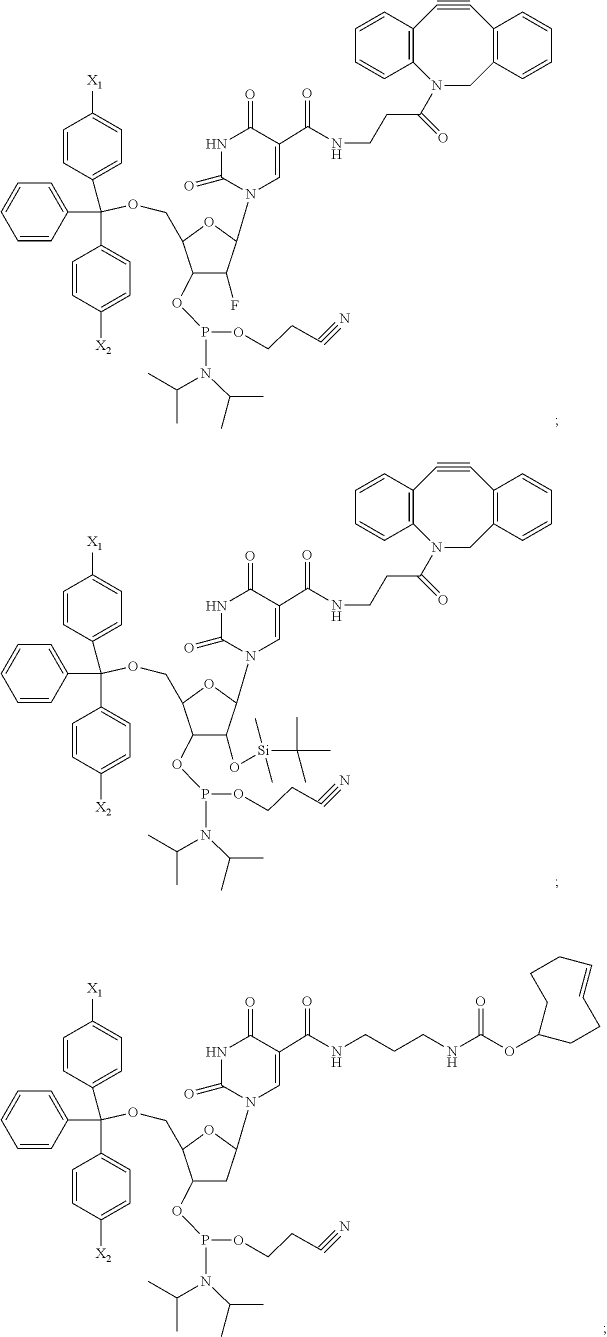

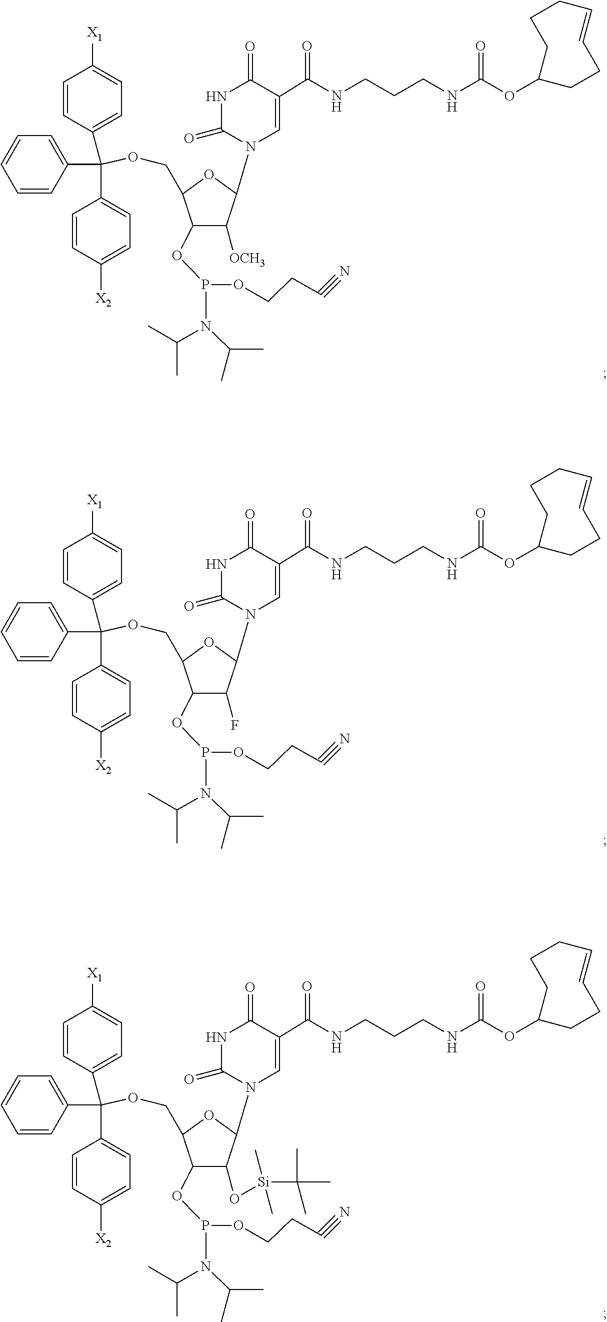

and salts thereof.

In some embodiments, a compound having the structure

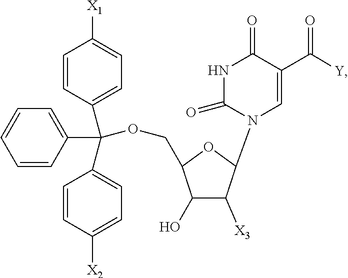

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy. In some embodiments, Y is

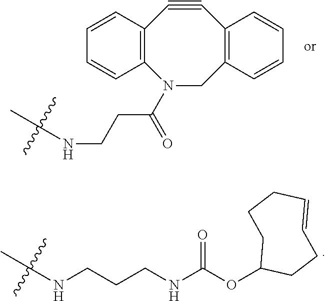 [SMILES: *NCCC(=O)N1Cc2ccccc2C#Cc2ccccc21 ; *NCCCNC(=O)OC1CC/C=C/CCC1]

In some embodiments, X₁ and X₂ are methoxy. In some embodiments, X₃ is hydrogen. In some embodiments, X₃ is methoxy. In some embodiments, X₃ is fluoro. In some embodiments, X₃ is tert-butyldimethylsilyloxy. In some embodiments, Y is

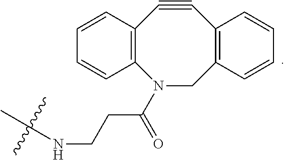 [SMILES: *NCCC(=O)N1Cc2ccccc2C#Cc2ccccc21]

In some embodiments, Y is

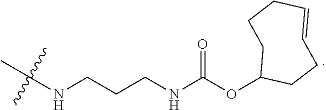 [SMILES: *NCCCNC(=O)OC1CC/C=C/CCC1]

In some embodiments, the compound is selected from:

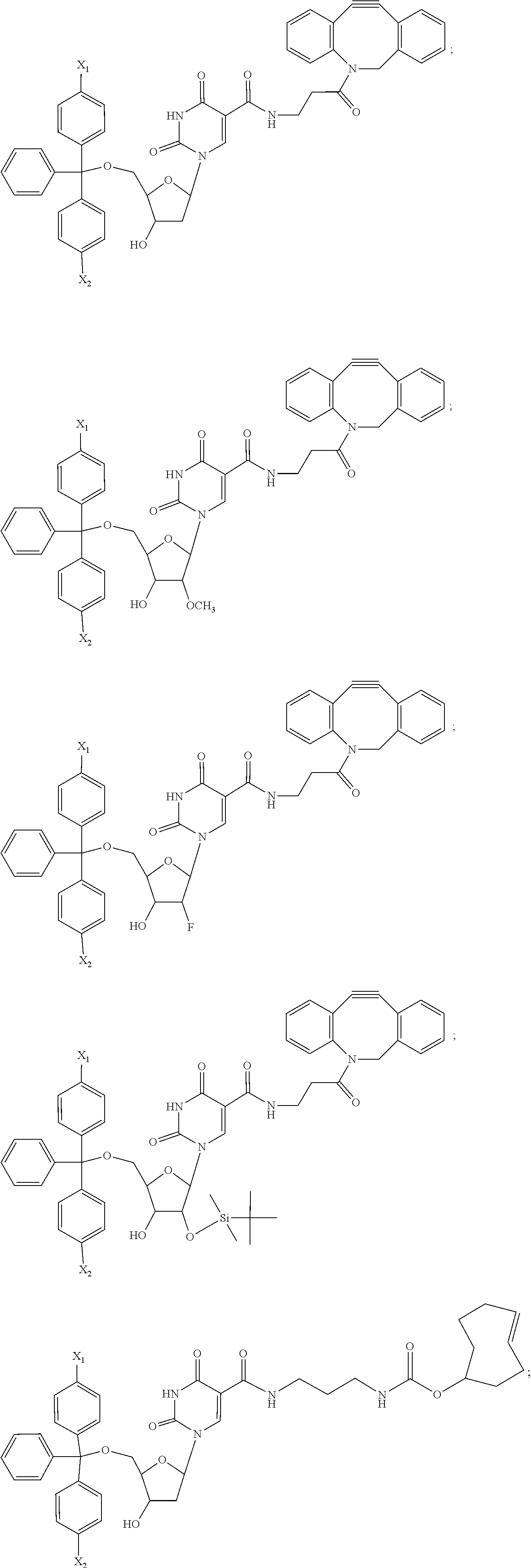

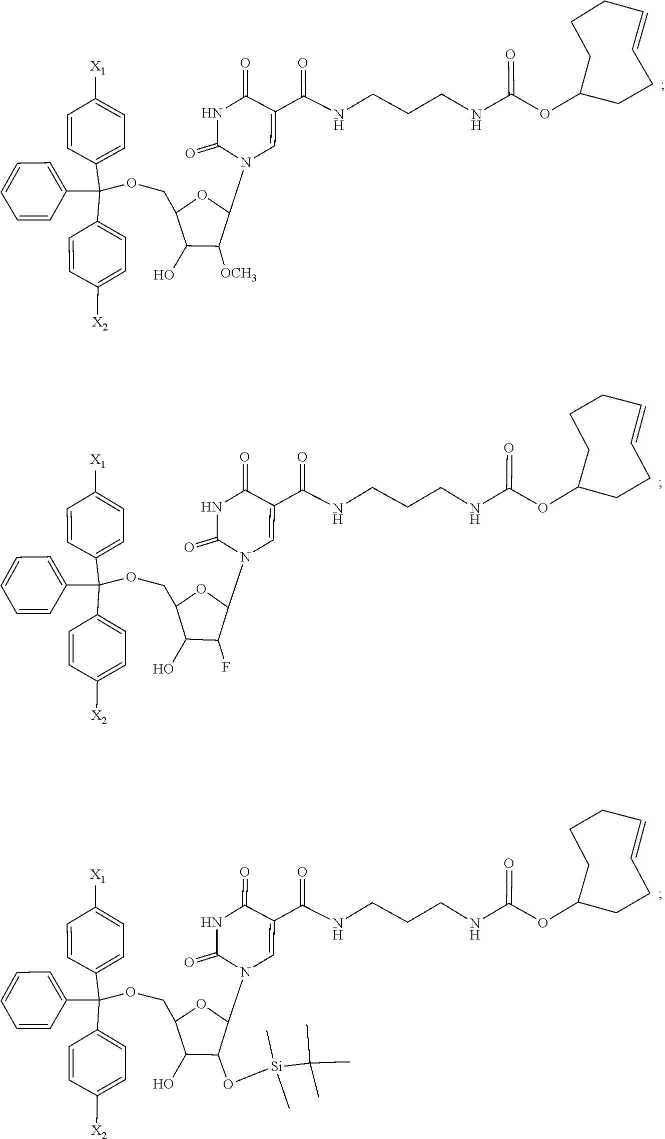

and salts thereof.

In some embodiments, a method of producing a compound having the structure:

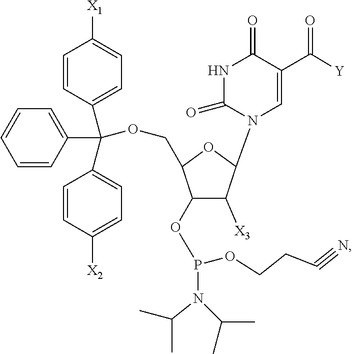

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy.

In some embodiments, Y is

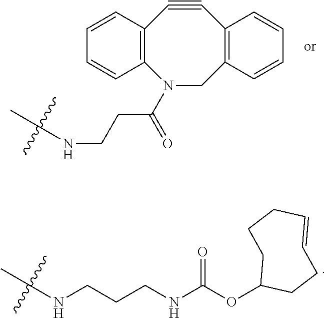 [SMILES: *NCCC(=O)N1Cc2ccccc2C#Cc2ccccc21 ; *NCCCNC(=O)OC1CC/C=C/CCC1]

In some embodiments, the method comprising reacting the compound

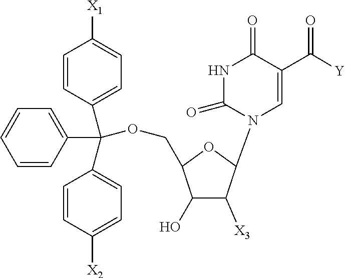

or a salt thereof, with 2-cyanoethyl N,N,N′,N′-tetraisopropylphosphorodiamidite and pyridine trifluoroacetic acid in dichloromethane, 2-cyanoethyl N,N-diisopropyl chlorophosphoramidite and diisopropylethylamine in dichloromethane, or similar conditions.

In some embodiments, the method produces a compound selected from:

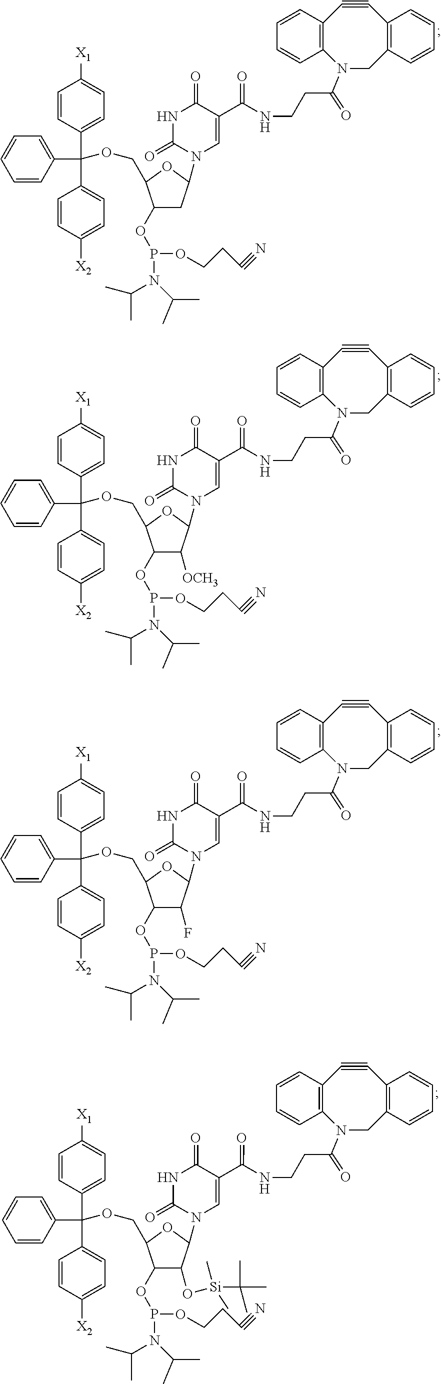

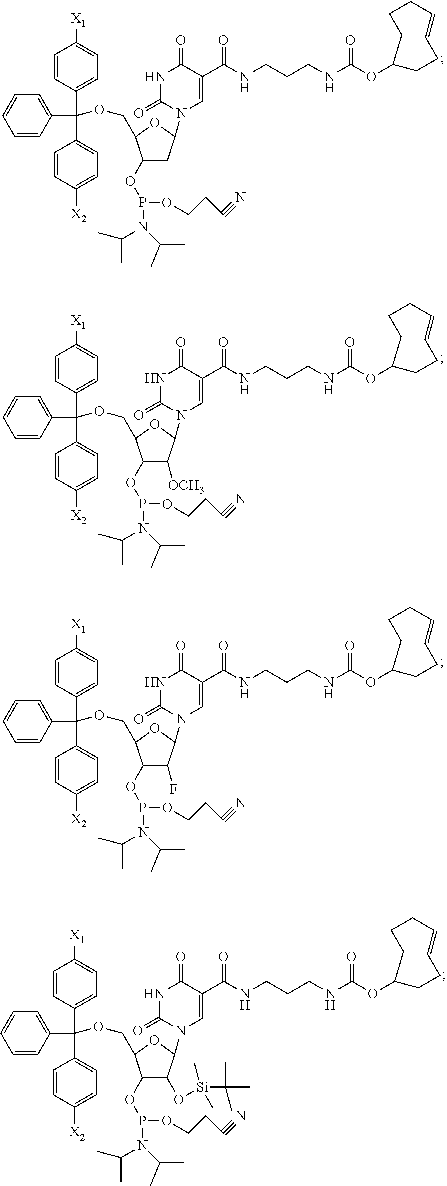

and salts thereof.

In some embodiments, a method of producing a compound having the structure:

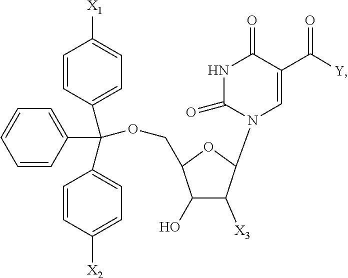

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy. In some embodiments, Y is

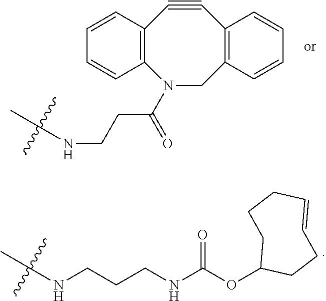 [SMILES: *NCCC(=O)N1Cc2ccccc2C#Cc2ccccc21 ; *NCCCNC(=O)OC1CC/C=C/CCC1]

In some embodiments, the method comprises reacting the compound

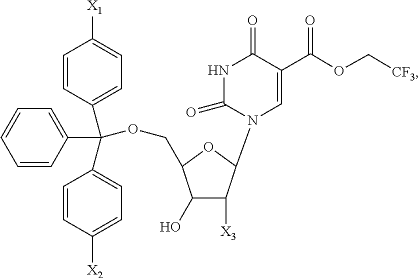

or a salt thereof, with the compound

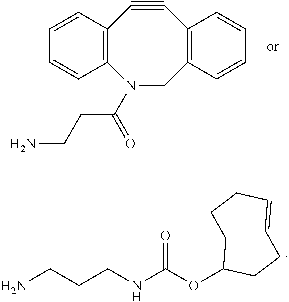 [SMILES: NCCC(=O)N1Cc2ccccc2C#Cc2ccccc21 ; NCCCNC(=O)OC1CC/C=C/CCC1]

In some embodiments, the method produces a compound selected from:

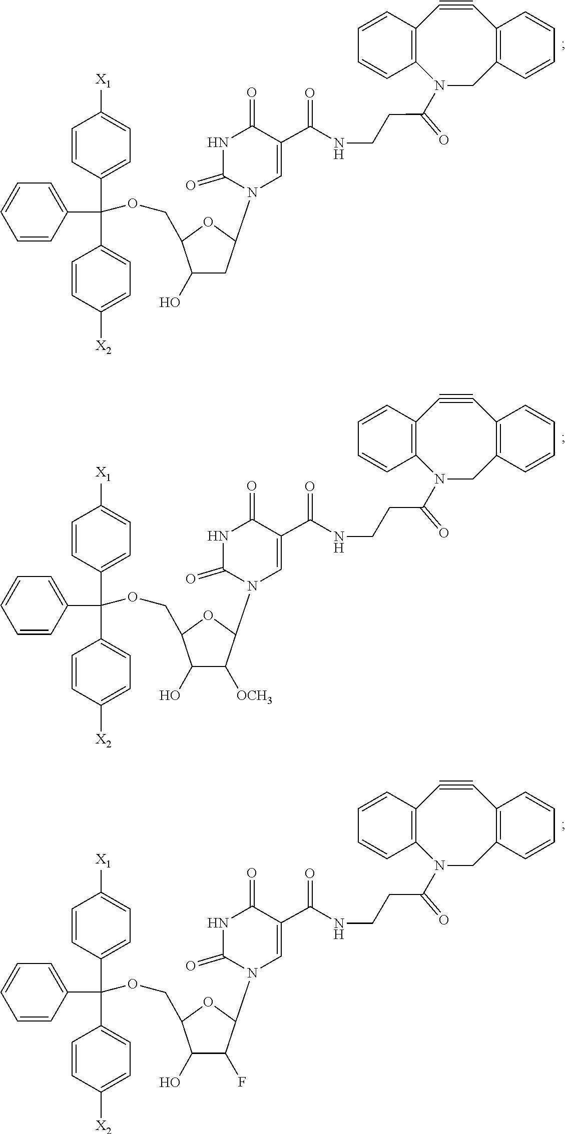

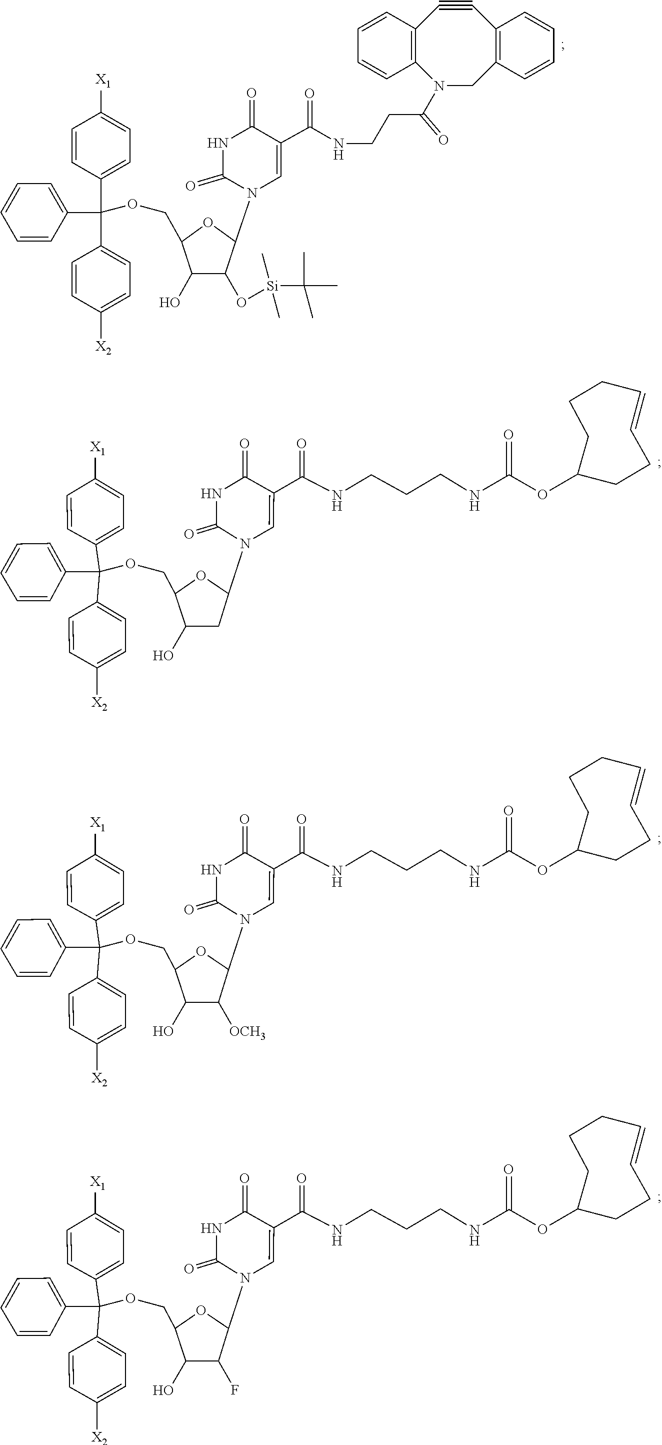

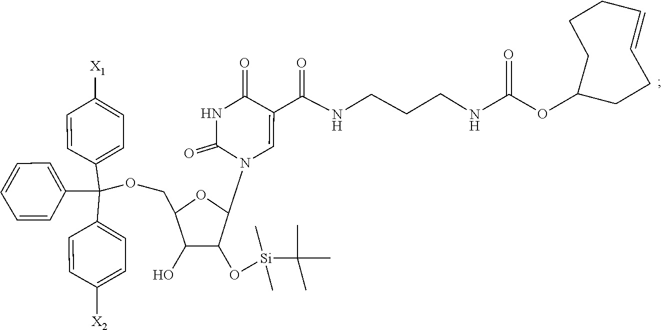

and salts thereof.

In some embodiments, a method of producing a compound having the structure:

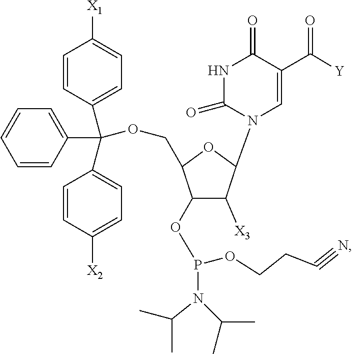

or a salt thereof, is provided,
wherein,
   X₁ and X₂ are each independently selected from methoxy and hydrogen;
   X₃ is selected from a methoxy, fluoro, hydrogen, and tert-butyldimethylsilyloxy; and
   Y is

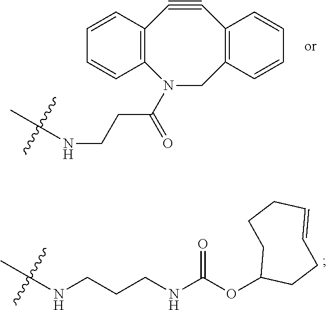 [SMILES: *NCCC(=O)N1Cc2ccccc2C#Cc2ccccc21 ; *NCCCNC(=O)OC1CC/C=C/CCC1]

comprising the steps of:
a) reacting the compound

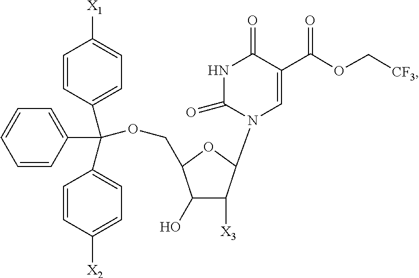

or a salt thereof, with the compound

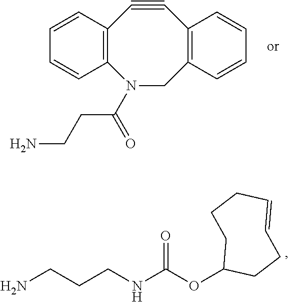 [SMILES: NCCC(=O)N1Cc2ccccc2C#Cc2ccccc21 ; NCCCNC(=O)OC1CC/C=C/CCC1]

to form the compound

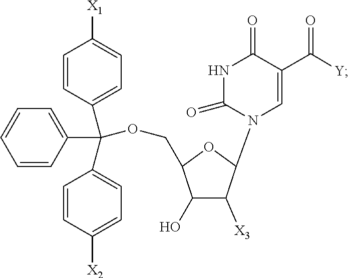

and
b) reacting the compound

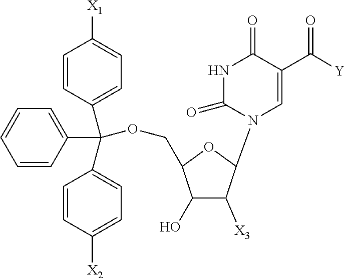

with 2-cyanoethyl N,N,N′,N′-tetraisopropylphosphorodiamidite and pyridine trifluoroacetic acid in dichloromethane, 2-cyanoethyl N,N-diisopropyl chlorophosphoramidite and diisopropylethylamine in dichloromethane, or similar conditions.

In some embodiments, the method produces a compound selected from:

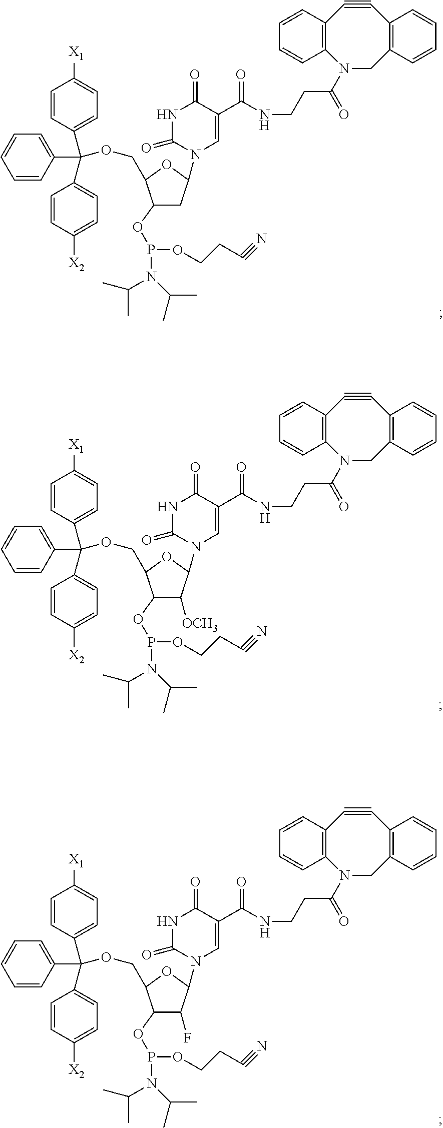

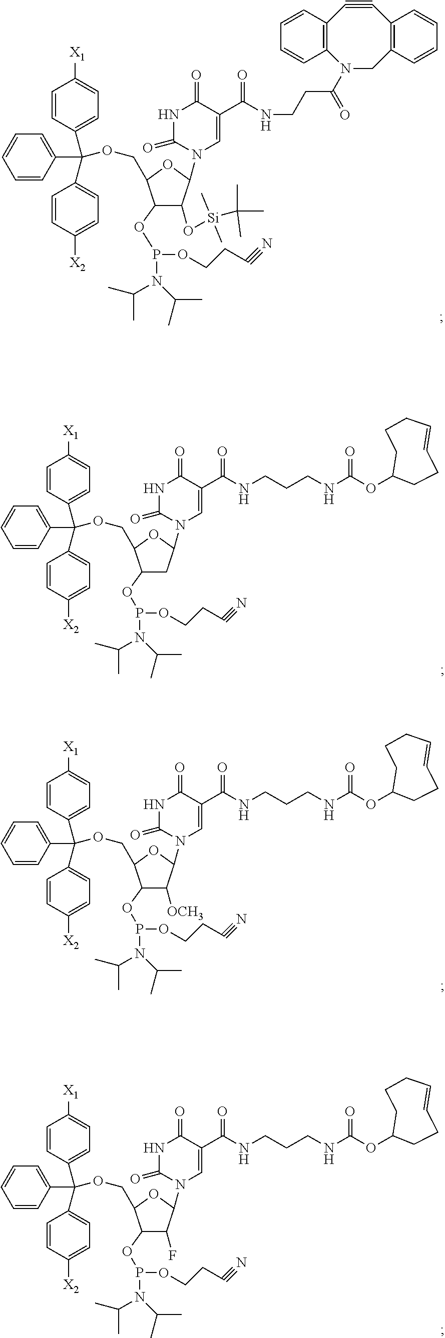

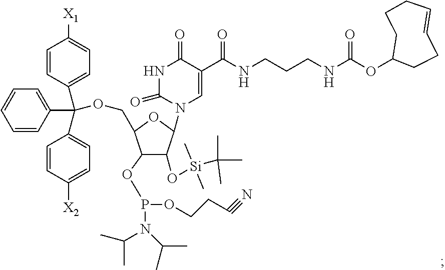

and salts thereof.

In embodiments, oligonucleotides are provided, comprising at least one spin-labeled nucleotide, wherein at least one spin-labeled nucleotide in the oligonucleotide has the structure:

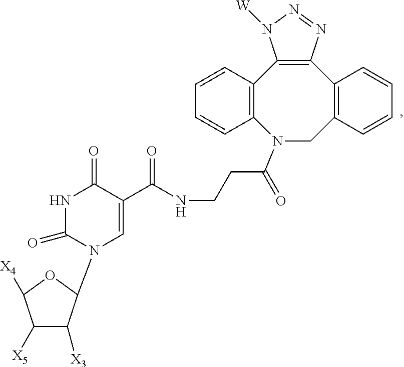

wherein W is a functional molecule. In some embodiments, X₃ is selected from a methoxy, fluoro, hydrogen, and tert-butyldimethylsilyloxy. In some embodiments, X₄ is selected from OH, —OR′, —SR′, and —Z—P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

In embodiments, oligonucleotides are provided, comprising at least one spin-labeled nucleotide, wherein at least one spin-labeled nucleotide in the oligonucleotide has the structure:

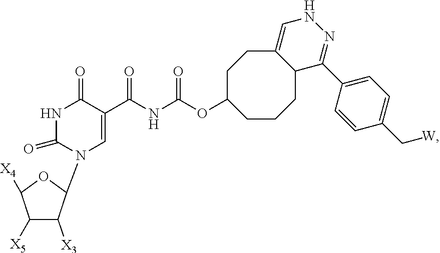

wherein W is a payload moiety. In some embodiments, X₃ is selected from a methoxy, fluoro, hydrogen, and tert-butyldimethylsilyloxy. In some embodiments, X₄ is selected from OH, —OR′, —SR′, and —Z—P(Z′)(Z″)O—R″, wherein Z, Z′, and Z″ are each independently selected from O and S, and R is an adjacent nucleotide in the oligonucleotide, R′ is H or a cap, and R″ is H, a cap, or an adjacent nucleotide. In some embodiments, X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

In some embodiments, a method of producing an oligonucleotide comprising at least one 5-position modified nucleoside is provided, comprising synthesizing an oligonucleotide comprising at least one nucleotide having the structure:

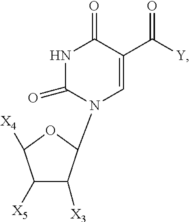

wherein Y is

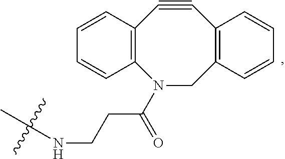 [SMILES: *NCCC(=O)N1Cc2ccccc2C#Cc2ccccc21]

into a nucleotide sequence on a solid support; and reacting the oligonucleotide with a reagent comprising a payload moiety and an azide moiety. In some embodiments, X₃ is selected from a methoxy, fluoro, hydrogen, and tert-butyldimethylsilyloxy. In some embodiments, X₄ is selected from OH, —OR′, —SR′, and —Z—P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

In some embodiments, a method of producing an oligonucleotide comprising at least one 5-position modified nucleoside is provided, comprising synthesizing an oligonucleotide comprising at least one nucleotide having the structure:

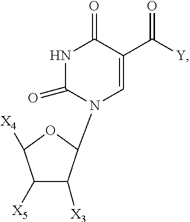

wherein Y is

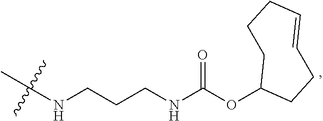 [SMILES: *NCCCNC(=O)OC1CC/C=C/CCC1]

into a nucleotide sequence on a solid support; and reacting the oligonucleotide with a reagent comprising a payload moiety and a tetrazine moiety. In some embodiments, X₃ is selected from a methoxy, fluoro, hydrogen, and tert-butyldimethylsilyloxy. In some embodiments, X₄ is selected from OH, —OR′, —SR′, and —Z—P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

### BRIEF DESCRIPTION OF THE FIGURES

FIG. 1 shows a chromatogram of DBCO-modified oligonucleotide overlaid with the same oligonucleotide clicked to TEMPO-azide (two peaks corresponding to two diastereomers).

FIG. 2 shows chromatograms of TCO-modified oligonucleotide (bottom panel), cyanine-3 tetrazine solution (middle panel) and the resulting product of the oligonucleotide clicked to the cyanine-3 tetrazine (top panel).

### DETAILED DESCRIPTION

In some embodiments, the compounds provided herein allow for the use of copper-free click chemistry reactions, which may have advantages over copper-requiring click reactions such as copper-catalyzed alkyne-azide cycloadditions. In some embodiments, omitting the copper catalyst may reduce or eliminate cell toxicity. See, e.g., Jewett et al., Chem Soc Rev, 2010, 39 (4), 1272-1279. In addition to improved biological compatibility, these copper-free reactions may be easier to control and/or optimize because the reaction involves fewer components. Moreover, purification may be more straightforward and may be carried out, in some embodiments, by desalting or size exclusion methods. In some embodiments, the compounds provided herein comprising a cyclooctyne at the five position of a uracil nucleobase allows for simpler, faster, and/or more readily controlled reactions with a tetrazine-payload in the absence of a copper catalyst.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean+20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs).

As used herein, the term “mod dU” is used to generally refer to uridylyl nucleotides comprising a 5-position modification. Use of the term “mod dU” is not intended to be limiting with regard to the 2′ position of the ribose, and the term should be construed to include, but not be limited to, nucleotides comprising, for example, —H, —OH, —Ome, or —F at the 2′-position, unless a particular 2′ moiety is indicated.

As used herein, the term “DBCO dU” is used to generally refer to uridylyl nucleotides comprising a 5-position dibenzocyclooctyne. Use of the term “DBCO dU” is not intended to be limiting with regard to the 2′ position of the ribose, and the term should be construed to include, but not be limited to, nucleotides comprising, for example, —H, —OH, —Ome, or —F at the 2′-position, unless a particular 2′ moiety is indicated.

As used herein, the term “TCO dU” is used to generally refer to uridylyl nucleotides comprising a 5-position trans-cyclooctene. Use of the term “TCO dU” is not intended to be limiting with regard to the 2′ position of the ribose, and the term should be construed to include, but not be limited to, nucleotides comprising, for example, —H, —OH, —OMe, or —F at the 2′-position, unless a particular 2′ moiety is indicated.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers, but the terms nucleic acid, oligonucleotide, and polynucleotide are not limited to aptamers.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, a “phorphoramidite” is a nucleotide comprising a

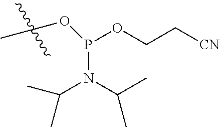 [SMILES: *OP(OCCC#N)N(C(C)C)C(C)C]

group attached to the 3′ carbon of the ribose, or an equivalent position on another sugar moiety. In some embodiments, a phosphoramidite comprises a protecting group on the 5′-OH of the ribose, such as a trityl protecting group, for example, a dimethoxytrityl protecting group.

As used herein, “solid phase synthesis” refers to solid-phase oligonucleotide synthesis using phosphoramidite chemistry, unless specifically indicated otherwise.

As used herein, “click chemistry reaction” or “click reaction” refers to bio-orthogonal reaction that joins two compounds together under mild conditions in a high yield reaction that generates minimal, biocompatible and/or inoffensive byproducts. In some embodiments, a click chemistry reaction requires a copper catalyst. In some embodiments, a click chemistry reaction is carried out in the absence of a copper catalyst. In some embodiments, a click chemistry reaction is a copper-free reaction. In some embodiments, a click chemistry reaction is a copper-free reaction and is promoted, for example, by ring strain.

### Compounds

The present disclosure provides the compounds shown in Table A, as well as salts thereof, and methods of making and using the compounds.

TABLE A
Compounds of the disclosure

| Compound | Name | Structure |
|---|---|---|
| 1 | DBCOdU CEP | 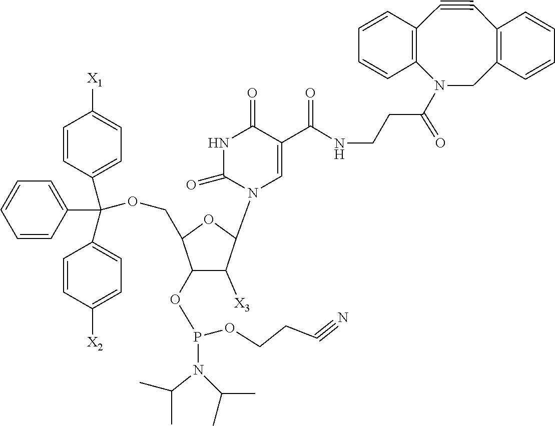 |
| 2 | TCOdU CEP | 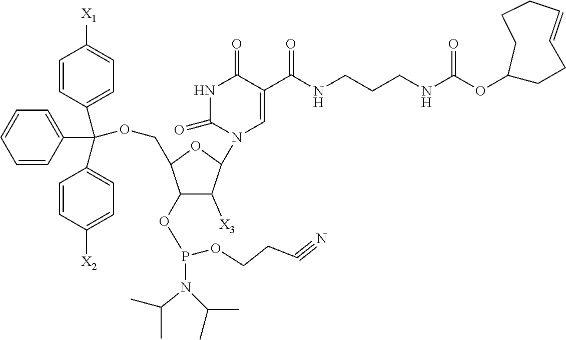 |
| 3 | DMT-DBCOdU | 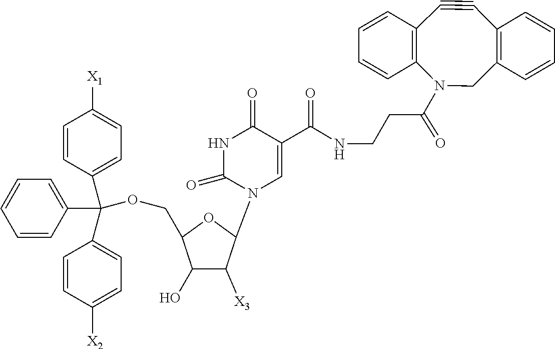 |
| 4 | DMT-TCOdU | 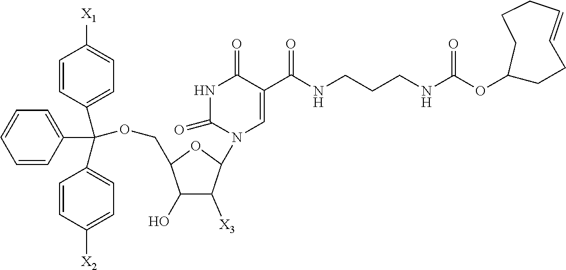 |

X₃ in the structures in Table A may, in some embodiments, be selected from methoxy, fluoro, hydrogen, and tert-butyldimethylsilyloxy. In some embodiments, compounds 1 to 4 in Table A may be used in solid-phase oligonucleotide synthesis to produce oligonucleotides comprising one or more spin-labeled nucleotides. Also provided herein are compounds comprising a structure selected from compounds 5 to 8, wherein the 3′ carbon of the ribose is linked to a solid phase, such as controlled-pore glass, through a linker moiety. In some embodiments, the 3′ carbon of the ribose is linked to a solid phase through a linker moiety selected from succinate, diglycolate, and alkylamino.

The compounds in Table A may be synthesized, in some embodiments, using the methods described herein, such as in the Examples herein.

### Salts

It may be convenient or desirable to prepare, purify, and/or handle a corresponding salt of the compound.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R^{X+}, NH₂R^{X}₂⁺, NHR^{X}₃⁺, NR^{X}₄⁺). Examples of some suitable substituted ammonium ions are those derived from: ethylamine, diethylamine, dicyclohexylamine, triethylamine, butylamine, ethylenediamine, ethanolamine, diethanolamine, piperizine, benzylamine, phenylbenzylamine, choline, meglumine, and tromethamine, as well as amino acids, such as lysine and arginine. An example of a common quaternary ammonium ion is N(CH₃)₄⁺.

If the compound is cationic or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Modified Oligonucleotides

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Nonlimiting exemplary caps include 5′-trimethoxystilbene cap, 5′ pyrene cap, 5′ adenylated cap, 5′ guanosine triphosphate cap, 5′ N7-methyl guanosine triphosphate cap, and 3′ Uaq cap. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

Oligonucleotides can also contain analogous forms of ribose or deoxyribose sugars that are generally known in the art, including 2′-O-methyl, 2′-O-allyl, 2′-O-ethyl, 2′-O-propyl, 2′-O—CH₂CH₂OCH₃, 2′-fluoro, 2′—NH₂ or 2′-azido, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NR^{X}₂ (“amidate”), P(O) R^{X}, P(O)OR^{X}′, CO or CH₂ (“formacetal”), in which each R^{X} or R^{X}′ are independently H or substituted or unsubstituted alkyl (C1-C20) optionally containing an ether (—O—) linkage, aryl, alkenyl, cycloalky, cycloalkenyl or araldyl. Not all linkages in an oligonucleotide need be identical. Substitution of analogous forms of sugars, purines, and pyrimidines can be advantageous in designing a final product, as can alternative backbone structures like a polyamide backbone, for example.

Oligonucleotides can also contain analogous forms of carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. An oligonucleotide can be further modified after polymerization, such as by conjugation with a labeling component.

### Preparation of Oligonucleotides

The automated synthesis of oligodeoxynucleosides is routine practice in many laboratories (see, e.g., Matteucci, M. D. and Caruthers, M. H., (1990) J. Am. Chem. Soc., 103:3185-3191, the contents of which are hereby incorporated by reference in their entirety). Synthesis of oligoribonucleosides is also well known (see e.g., Scaringe, S. A., et al., (1990) Nucleic Acids Res. 18:5433-5441, the contents of which are hereby incorporated by reference in their entirety). As noted herein, the phosphoramidites are useful for incorporation of the modified nucleoside into an oligonucleotide by chemical synthesis, and the triphosphates are useful for incorporation of the modified nucleoside into an oligonucleotide by enzymatic synthesis. (See e.g., Vaught, J. D. et al. (2004) J. Am. Chem. Soc., 126:11231-11237; Vaught, J. V., et al. (2010) J. Am. Chem. Soc. 132, 4141-4151; Gait, M. J. “Oligonucleotide Synthesis a practical approach” (1984) IRL Press (Oxford, UK); Herdewijn, P. “Oligonucleotide Synthesis” (2005) (Humana Press, Totowa, N.J. (each of which is incorporated herein by reference in its entirety).

In some embodiments, the compounds provided herein, and in particular, compounds of Table A, may be used in standard phosphoramidite oligonucleotide synthesis methods, including automated methods using commercially available synthesizers. Following synthesis, the click chemistry moiety on the oligonucleotide can be reacted with a payload reagent modified with a complementary click chemistry moiety to yield mod dU. An exemplary click reaction used in the present disclosure is strain-promoted alkyne-azide cycloaddition. Another exemplary click reaction used in the present disclosure is trans-cyclooctene-tetrazine ligation. Various reagents comprising a payload moiety and an azide moiety or a tetrazine moiety for use in click chemistry are commercially available.

### EXAMPLES

The following examples are presented in order to more fully illustrate some embodiments of the invention. They should, in no way be construed, however, as limiting the broad scope of the invention. Those of ordinary skill in the art can readily adopt the underlying principles of this discovery to design various compounds without departing from the spirit of the current invention.

### Example 1: Synthesis of DBCO-Labeled Phosphoramidites

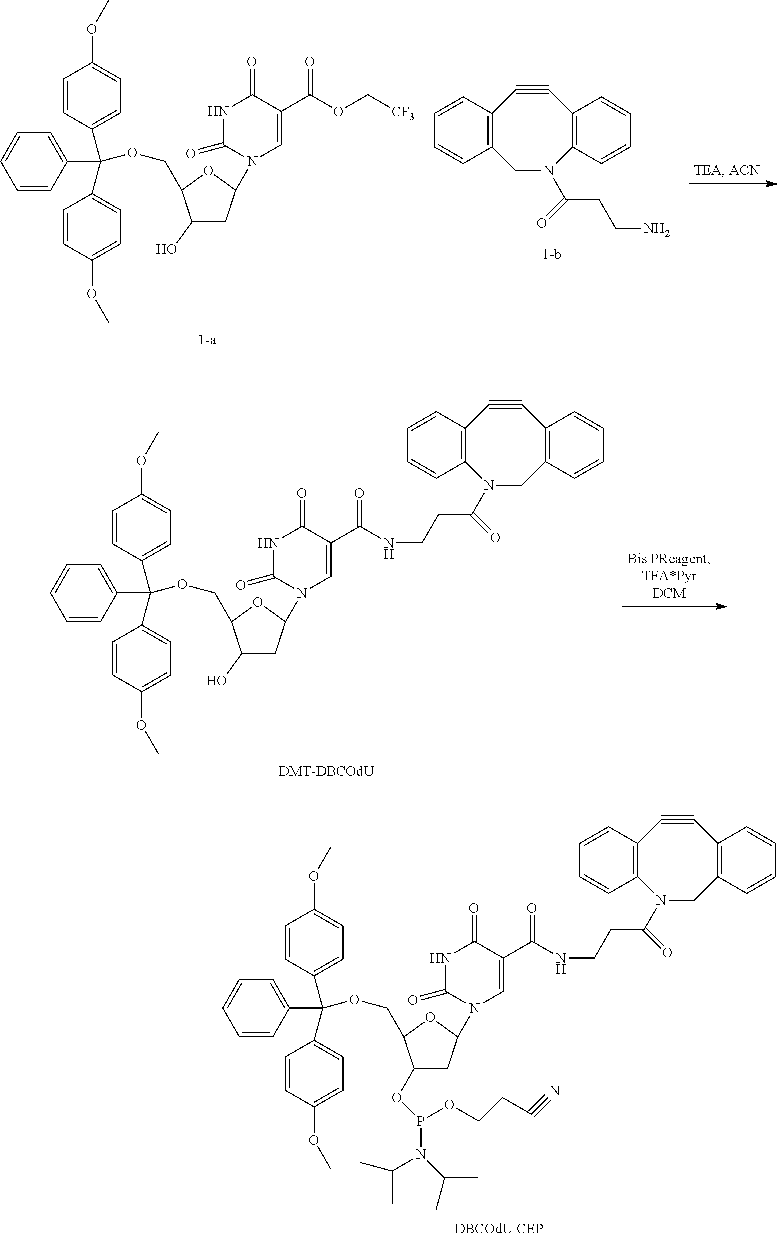

### Example S1.1 Synthesis of DMT-DBCOdU

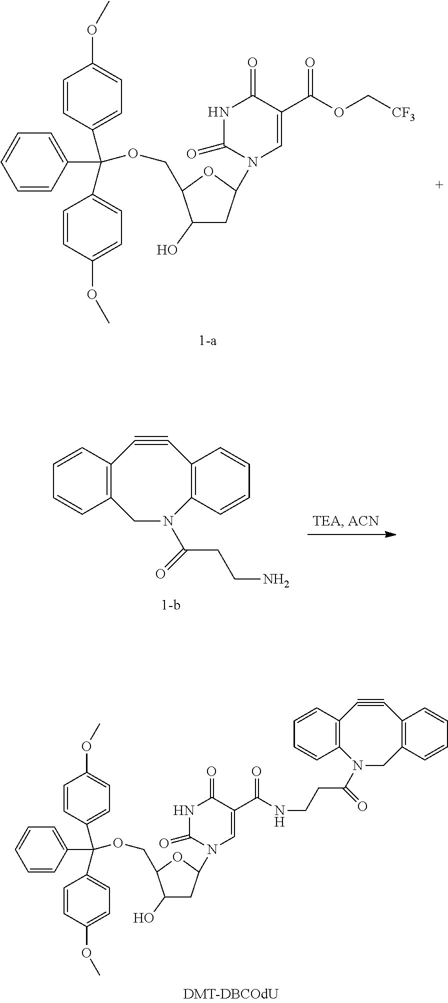 [SMILES: COc1ccc(C(OCC2OC(n3cc(C(=O)OCC(F)(F)F)c(=O)[nH]c3=O)CC2O)(c2ccccc2)c2ccc(OC)cc2)cc1 ; NCCC(=O)N1Cc2ccccc2C#Cc2ccccc21 ; COc1ccc(C(OCC2OC(n3cc(C(=O)NCCC(=O)N4Cc5ccccc5C#Cc5ccccc54)c(=O)[nH]c3=O)CC2O)(c2ccccc2)c2ccc(OC)cc2)cc1]

The starting material, 5′-O-dimethoxytrityl-5-trifluoroethoxycarbonyl-2′-deoxyuridine (Scheme 1, product 1-a, 9.5, 14.5 mmol; prepared as previously reported, e.g., in Nomura et al. Nucleic Acids Research, 1997, 25, 2784-2791; Ito et al. Nucleic Acids Research, 2003, 25, 2514-2523) was charged into a dry, argon-purged round bottomed flask. Dry acetonitrile (42 mL) and dibenzocyclooctyneamine (Scheme 1, product 1-b, 4.8 g, 17.4 mmol, 1.2 eq) were added to the flask and the mixture was stirred to dissolve the solids. Triethylamine (4.0 mL, 29.0 mmol, 2 eq) was added to the stirring mixture, which was transferred to an oil bath and was heated under an inert atmosphere at 65° C. After stirring approximately 5 hours, solids had precipitated out of solution forming thick slurry. The mixture was allowed to cool to room temperature before filtering. White solids were washed with acetonitrile and dried in desiccator under vacuum. (8.76 g, 72% yield).

¹H-NMR (400 mHz, DMSO-d6): δ=11.78 (s (b), 1H), 8.58 (q, J=5.6 Hz, 1H), 8.29 (d, J=1.2 Hz, 1H), 7.61 (d, J=8.0 Hz, 1H) 7 . . . 45-7.52 (m, 1H), 7.44-7.27 (m, 7H), 7.18-7.26 (m, 7H), 6.82 (dd, J_{A}=8.8, J_{B}=2.1 Hz, 4H), 6.02 (t, J=6.4 Hz, 1H), 5.78-5.33 (m, 1H), 5.01 (dd, J_{A}=15.6, J_{B}=2.1 Hz, 1H), 4.0-4.10 (m, 1H), 3.84-3.92 (m, 1H), 3.65 (d, J=4.4, 5H), 3.58 (d, J=14 Hz, 1H), 3.15-3.25 (m, 2H), 3.08-3.15 (m, 2H), 2.047-2.27 (m, 2H), 1.79-1.95 (m, 1H).

¹³C-NMR (100 mHz, CD₃CN): δ=170.7 (1C), 163.26 (1C), 161.6 (1C), 158.45 (1C), 158.42 (1C), 151.69 (1C), 149.87 (1C), 148.73 (1C), 145.72 (1C), 145.68 (1C), 145.29 (1C), 135.88 (1C), 135.74 (1C), 132.80 (1C), 130.20 (2C), 130.07 (2C), 129.92 (1C), 129.29 (1C), 128.60 (1C), 128.49 (1C), 128.25 (2C), 128.12 (1C), 128.05 (2C), 127.20 (1C), 127.03 (1C), 125.61 (1C), 122.89 (1C), 121.86 (1C), 114.74 (1C), 113.63 (4C), 108.42 (1C), 105.57 (1C), 86.28 (1C), 86.22 (1C), 86.12 (1C), 70.74 (1C), 64.03 (1C), 55.36 (1C), 55.24 (1C), 35.39 (1C), 36.66 (1C). MS (m/z) calcd for C₄₉H₄₄N₄O₉, 832.91; found 831.3 [M−H]⁻ (ESI⁻).

### Example S1.2 Synthesis of DBCOdU CEP

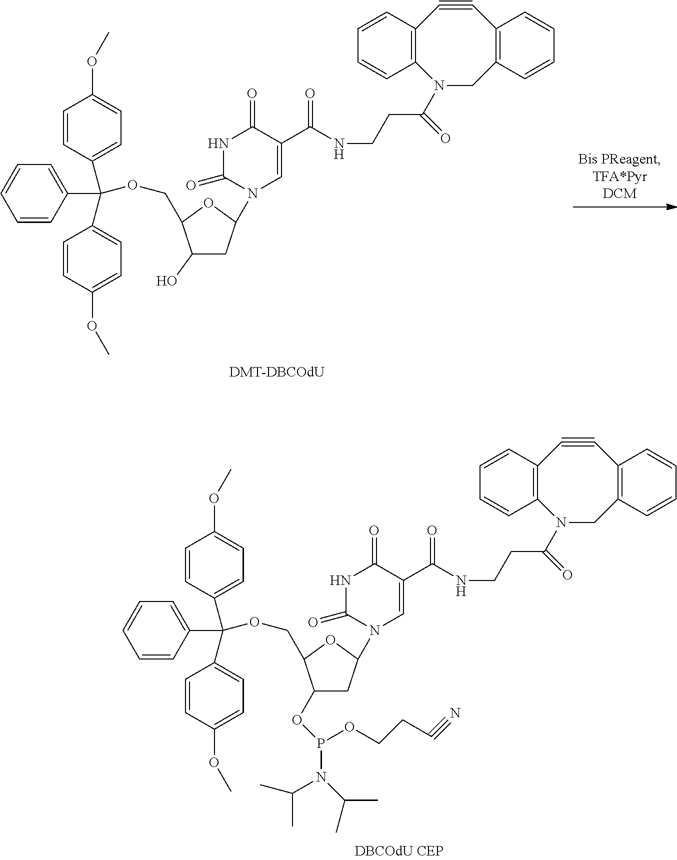 [SMILES: COc1ccc(C(OCC2OC(n3cc(C(=O)NCCC(=O)N4Cc5ccccc5C#Cc5ccccc54)c(=O)[nH]c3=O)CC2O)(c2ccccc2)c2ccc(OC)cc2)cc1 ; COc1ccc(C(OCC2OC(n3cc(C(=O)NCCC(=O)N4Cc5ccccc5C#Cc5ccccc54)c(=O)[nH]c3=O)CC2OP(OCCC#N)N(C(C)C)C(C)C)(c2ccccc2)c2ccc(OC)cc2)cc1]

In a round-bottomed flask with magnetic stirring, the product of the previous step (Scheme 1, product 3, 8.76 g, 10.5 mmol) was slurried in anhydrous dichloromethane (30 mL) under argon. To the reaction mixture was added 2-cyanoethyl-N,N,N′,N′-tetraisopropylphosphine (Bis Reagent, 3.5 mL, 11.0 mmol, 1.05 eq) followed by pyridine trifluoroacetate (2.23 g, 11.5 mmol, 1.1 eq). Upon addition of 2-cyanoethyl-N,N,N′,N′-tetraisopropylphosphine and pyridine trifluoroacetate, the starting material dissolved completely. The reaction was stirred for 1.25 hours, then the crude mixture was applied to a silica gel flash column equilibrated with 80% ethyl acetate/19% hexanes/1% triethylamine and product elution was achieved using increasing concentrations of ethyl acetate, with the final fractions being eluted using 100% ethyl acetate. All mobile phases were chilled to 0° C. and sparged with argon and product was collected into argon-purged bottles. Product-containing fractions were concentrated to provide a white to off-white foam (9.32 g, 86% yield).

¹H-NMR (400 mHz, DMSO-d₆): δ=11.8 (bs, 1H), 8.57 (t, J=5.7 Hz, 1H), 8.34 (d, J=5.6 Hz, 1H), 7.27-7.57 (m, 7H), 7.10-7.26 (m, 7H), 6.75-6.86 (m, 4H), 6.03 (m, 1H), 5.01 (d, J=14 Hz, 1H), 4.24-4.37 (m, 1H), 3.99-4.07 (m, 1H), 3.61-3.73 (m, 6H), 3.36-3.61 (m, 4H), 3.10-3.27 (m, 3H), 2.71 (t, J=6.0 Hz, 1H), 2.60 (td, J_{A}=6.0, J_{B}=0.8 Hz, 1H), 2.26-2.41 (m, 2H), 1.82-1.93 (m, 1H), 1.06 (dd, J_{A}=12.3, J_{B}=6.6 Hz, 8H), 0.91 (d, J=6.8 Hz, 2H).

³¹P-NMR (400 mHz, DMSO-d₆): δ=147.23/147.58 (d, J_{A}=1.6, J_{B}=3.7 Hz, 1P). MS (m/z) calcd for C₅₈H₆₁N₆O₁₀P, 1033.13; found 1031.4 [M−H]⁻ (ESI⁻).

### Example 2: Synthesis of TCO-Labeled Phosphoramidites

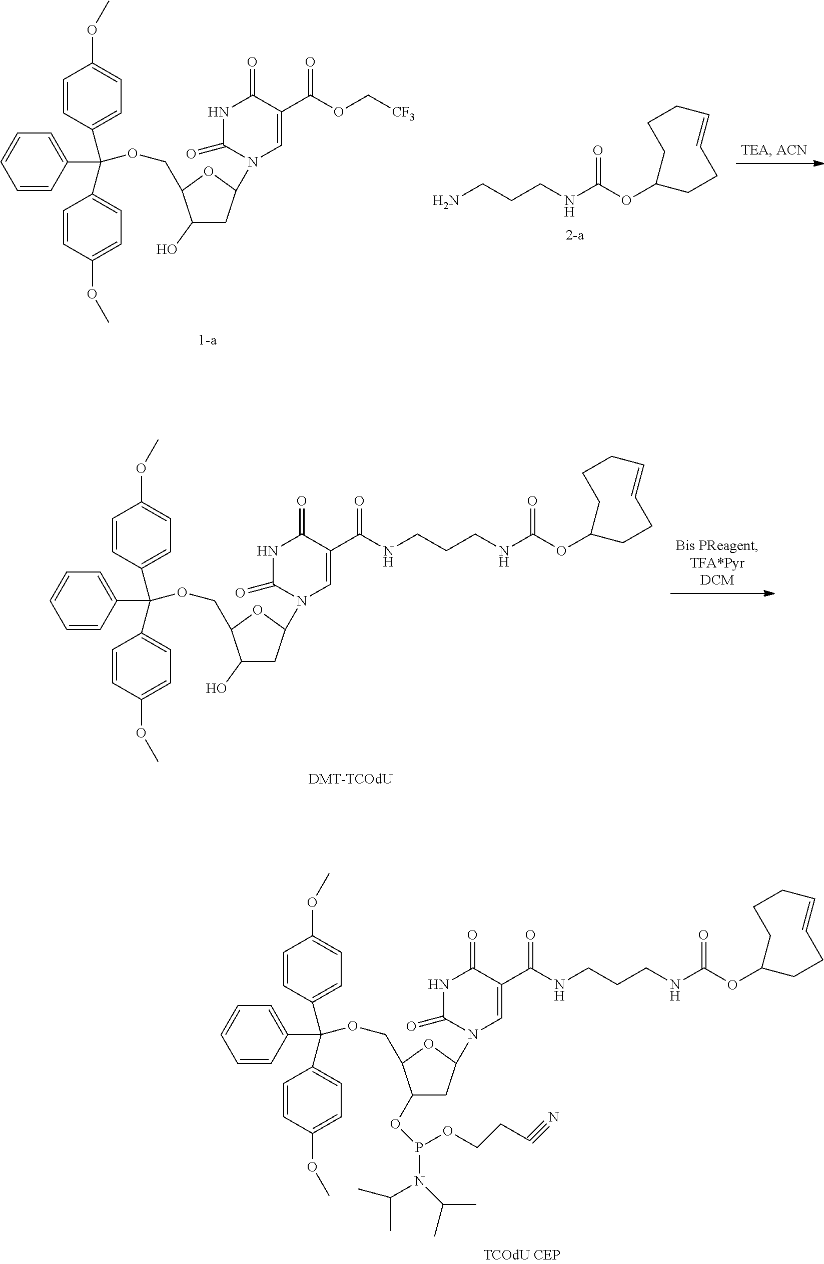 [SMILES: COc1ccc(C(OCC2OC(n3cc(C(=O)OCC(F)(F)F)c(=O)[nH]c3=O)CC2O)(c2ccccc2)c2ccc(OC)cc2)cc1 ; NCCCNC(=O)OC1CC/C=C/CCC1 ; COc1ccc(C(OCC2OC(n3cc(C(=O)NCCCNC(=O)OC4CC/C=C/CCC4)c(=O)[nH]c3=O)CC2O)(c2ccccc2)c2ccc(OC)cc2)cc1 ; COc1ccc(C(OCC2OC(n3cc(C(=O)NCCCNC(=O)OC4CC/C=C/CCC4)c(=O)[nH]c3=O)CC2OP(OCCC#N)N(C(C)C)C(C)C)(c2ccccc2)c2ccc(OC)cc2)cc1]

### Example S2.1 Synthesis of DMT-TCOdU

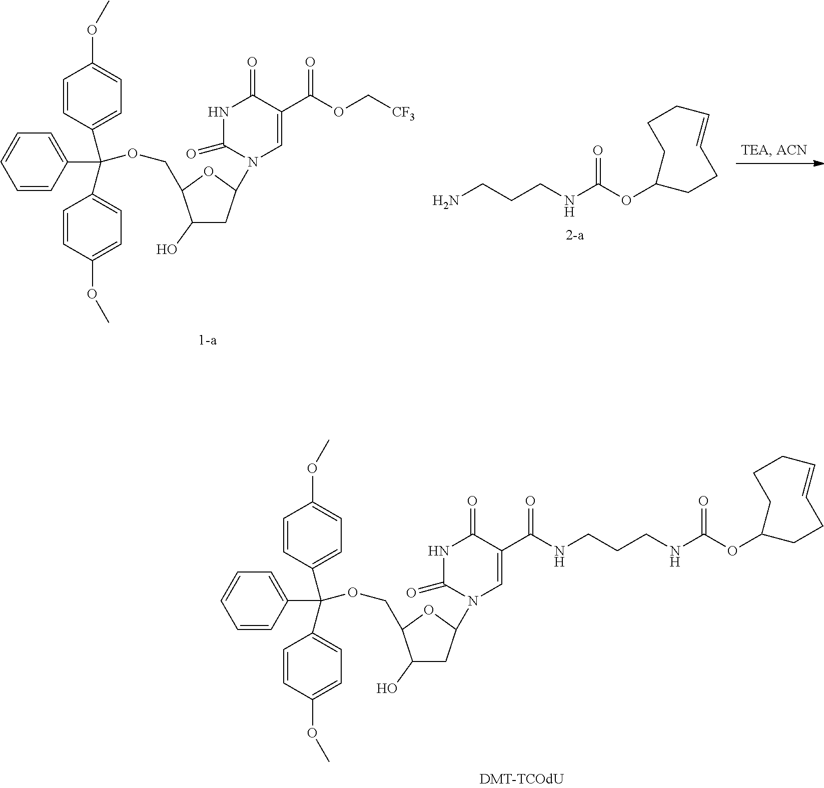 [SMILES: COc1ccc(C(OCC2OC(n3cc(C(=O)OCC(F)(F)F)c(=O)[nH]c3=O)CC2O)(c2ccccc2)c2ccc(OC)cc2)cc1 ; NCCCNC(=O)OC1CC/C=C/CCC1 ; COc1ccc(C(OCC2OC(n3cc(C(=O)NCCCNC(=O)OC4CC/C=C/CCC4)c(=O)[nH]c3=O)CC2O)(c2ccccc2)c2ccc(OC)cc2)cc1]

The starting material, 5′-O-dimethoxytrityl-5-trifluoroethoxycarbonyl-2′-deoxyuridine (Scheme 2, product 1-a, 2.08 g, 3.17 mmol)) was charged into a dry, argon-purged round bottomed flask. Dry acetonitrile (4 mL) and [(4E)-1-cylcooct-4-enyl]-N-(3-aminoproyl carbamate) hydrochloride (Scheme 2, product 1-b, 1.0 g, 3.81 mmol, 1.2 eq) were added to the flask and the mixture was stirred to dissolve the solids. Triethylamine (1.3 mL, 9.5 mmol, 3 eq) was added to the stirring mixture, which was transferred to a water bath and was heated under an inert atmosphere at 65° C. Reaction progress was monitored by reversed phase HPLC (Waters 2795 HPLC with a 2489 detector and using a Waters Symmetry column, buffer A: 100 mM triethylammonium acetate, buffer B: acetonitrile, gradient: 70% buffer B, isocratic, over 30 minutes). After stirring approximately 5 hours, analysis showed the reaction to be complete. The mixture was stirred at room temperature an additional 16 hours, when stirring was discontinued and solvent was evaporated to recover a yellowish foam. The crude mixture was applied to a silica gel flash column equilibrated with 1% triethylamine/75% ethyl acetate/24% hexanes. The product was initially eluted with the same mobile phase, which was modified as the elution progressed to 99% ethyl acetate/1% triethylamine and finally 2% methanol/97% ethyl acetate/1% triethylamine to complete the elution. Product-containing fractions were concentrated to provide a white to off-white foam (11.58 g, 91% yield).

¹H-NMR (300 mHz, DMSO-d6): δ=11.92 (s, 1H), 8.67 (t, J=5.6 Hz, 1H), 8.39 (s, 1H), 7.30-7.35 (m, 2H), 7.14-7.24 (m, 6H), 6.95 (t, J=5.6 hz, 1H), 6.84 (dd, J_{A}=9.0, J_{B}=2.1 Hz, 4H), 6.04 (t, J=6.4 Hz, 1H), 5.48-5.78 (m, 1H), 5.34-5.44 (m, 1H), 5.31 (d, J=4.4 Hz, 1H), 4.12-4.20 (m, 1H), 4.03-4.09 (m, 1H), 3.86 (q, J=4.4, 1H), 3.30 (s, 2H), 3.2 (q, J=7.0, 2H), 3.14 (d, J=4.4 Hz, 2H) 2.86-2.97 (m, 2H), 2.11-2.27 (m, 4H), 1.74-1.90 (m, 4H), 1.41-1.65 (m, 5H).

¹³C-NMR (100 mHz, DMSO-d6): δ=163.52 (1C), 161.82 (1C), 158.46 (2C), 156.20 (1C), 149.86 (1C), 145.77 (1C), 145.92 (1C), 135.90 (1C), 135.78 (1C), 135.34 (1C), 132.95 (1C), 130.21 (2C), 130.09 (2C), 128.26 (2C), 128.06 (2C), 127.04 (1C), 113.64 (4C), 105.70 (1C), 86.23 (1C), 86.22 (1C), 86.14 (1C), 79.39 (1C), 70.74 (1C), 64.03 (1C), 55.39 (2C), 41.12 (1C), 38.65 (1C), 38.17 (1C), 36.54 (1C), 34.18 (1C), 32.58 (1C), 31.01 (1C), 30.16 (1C). MS (m/z) calcd for C₄₃H₅₀N₄O₁₀, 782.89; found 781.3 [M−H]⁻ (ESI⁻).

### Example S2.2 Synthesis of TCOdU CEP

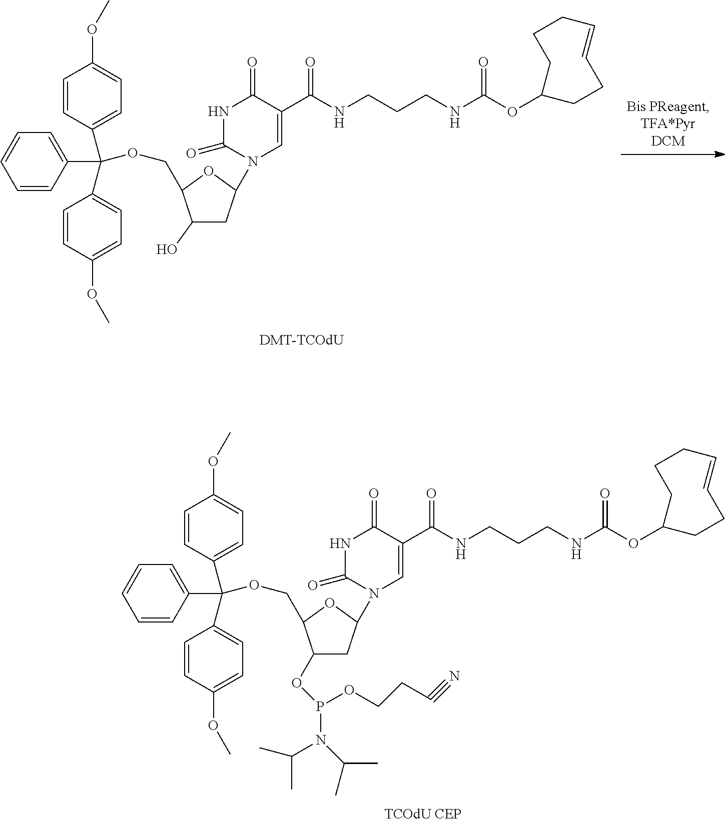 [SMILES: COc1ccc(C(OCC2OC(n3cc(C(=O)NCCCNC(=O)OC4CC/C=C/CCC4)c(=O)[nH]c3=O)CC2O)(c2ccccc2)c2ccc(OC)cc2)cc1 ; COc1ccc(C(OCC2OC(n3cc(C(=O)NCCCNC(=O)OC4CC/C=C/CCC4)c(=O)[nH]c3=O)CC2OP(OCCC#N)N(C(C)C)C(C)C)(c2ccccc2)c2ccc(OC)cc2)cc1]

In a round-bottomed flask with magnetic stirring, the product of the previous step (Scheme 2, DMT-TCOdU, 1.781 g, 2.27 mmol) was dissolved in anhydrous dichloromethane (6.5 mL) under argon. To the reaction mixture was added 2-cyanoethyl-N,N,N′,N′-tetraisopropylphosphine (Bis Reagent, 0.76 mL, 2.38 mmol, 1.05 eq) followed by pyridine trifluoroacetate (0.48 g, 2.5 mmol, 1.1 eq). The reaction was stirred for 1 hour, then analyzed by thin-layer chromatography (silica gel, eluent: 80% ethyl acetate/2% hexanes), which showed the reaction was complete. A silica gel chromatography column was prepared and conditioned with 80% ethyl acetate/19% hexanes/1% triethylamine followed by a wash of 80% ethyl acetate/20% hexanes. The crude mixture was applied to the prepared column and product elution was achieved using 80% ethyl acetate/20% hexanes followed by 100% ethyl acetate. All mobile phases were chilled to 0° C. and sparged with argon and product was collected into argon-purged bottles. Product-containing fractions were concentrated to provide a white to off-white foam (1.99 g, 89.2% yield).

¹H-NMR (300 mHz, DMSO-d₆): δ=11.88 (s (b), 1H), 8.68 (t, J=5.6 Hz, 1H), 8.43/8.42 (s, 1H), 7.30-7.36 (m, 2H), 7.12-7.28 (m, 6H), 6.94 (t, J=5.6 Hz, 1H), 6.83 (dd, J_{A}=9.0, J_{B}=2.4 Hz, 4H), 6.01-6.08 (m, 5H), 5.47-5.58 (m, 1H), 5.33-5.44 (m, 1H), 4.26-4.38 (m, 1H), 4.11-4.21 (m, 1H), 3.98-4.08 (m, 1H), 3.69/3.68 (s, 6H), 3.36-3.61 (m, 4H), 3.10-3.27 (m, 3H), 2.71 (t, J=6.0 Hz, 1H), 2.59 (td, J_{A}=6.0, J_{B}=0.8 Hz, 1H), 2.26-2.41 (m, 2H), 1.82-1.93 (m, 1H), 1.05 (dd, J_{A}=12.3, J_{B}=6.6 Hz 8H), 0.91 (d, J=6.8 Hz, 2H).

³¹P-NMR (300 mHz, DMSO-d₆): δ=147.22/147.58 (s, 1P). MS (m/z) calcd for C₅₂H₆₇N₆O₁₁P, 983.11; found 981.3 [M−H]⁻ (ESI⁻).

### Example 3: Solid-Phase Oligonucleotide Synthesis Using DBCO-Labeled Phosphoramidites

An ABI 3900 automated DNA synthesizer (Applied Biosystems, Foster City, CA) was used with conventional phosphoramidite methods with minor changes to the coupling conditions for modified phosphoramidites. Modified phosphoramidites were used in 0.1 M solutions using acetonitrile with 0-40% dichloromethane and 0-20% sulfolane as the solvent. Solid support was an ABI style fritted column packed with controlled pore glass (CPG, LGC Biosearch Technologies, Petaluma CA) loaded with 3′-DMT-dT succinate with 1000 Å pore size. All syntheses were performed at the 50 nmole scale and the 5′ end of each sequence was modified with a hexaethyleneglycol spacer and biotin group for support attachment. Introduction of a DBCO dU variant was done as a single-base replacement at select sites within the DNA strand using phosphoramidites synthesized according to Example 1. Deprotection was accomplished by treating with concentrated ammonium hydroxide at 55° C. for 4-6 hours, the product mixtures were filtered and residual solvents removed in a Genevac HT-12 evaporator. Identity and percent full length product were determined using an Agilent 1290 Infinity with an Agilent 6130B single quadrupole mass spectrometry detector using an Acquity C18 column 1.7 μm 2.1×100 mm (Waters Corp, Milford, MA).

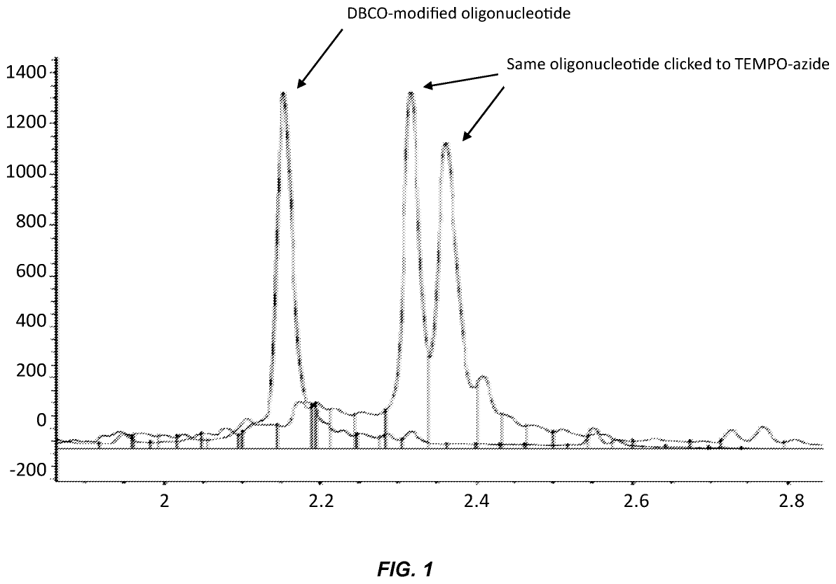

The resulting crude DBCO-modified oligonucleotide residues were then redissolved in Water for Injection (WFI, HyPure WFI Quality Water, HyClone Laboratories, Logan, UT, or similar) to 0.17 mM concentration (based on synthesis scale). A 100 mM solution of commercially sourced 4-azido-2,2,6,6-tetramethyl-piperidinyl-1-oxyl (TEMPO) azide (Glen Research cat #50-2007-92) was prepared in dimethyl sulfoxide. Each oligonucleotide mixture received an aliquot of the azide solution at a 4:1 ratio of azide to oligonucleotide (based on synthesis scale) and the resulting mixture was mixed at room temperature for 24 to 65 hours, at which time analysis by LC/MS (Agilent 1290 Infinity, configured as above) confirmed that each reaction had reached a quantitative cycloaddition. See FIG. 1. The resulting products had two stereoisomers which can be observed on the resulting chromatogram as dual peaks. See FIG. 1. Each reaction mixture was then applied to a centrifugal filter (Millipore Amicon Ultra-15 3K), washed three times with 5 mL WFI per wash for removal of small molecule impurities. Product was collected in approximately 500 μL WFI without further purification.

### Example 4: Solid-Phase Oligonucleotide Synthesis Using TCO-Labeled Phosphoramidites

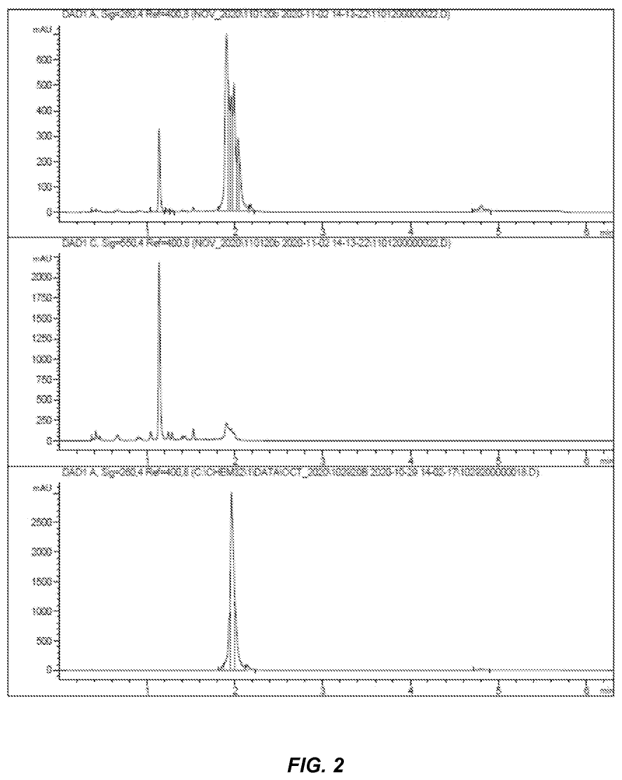

An ABI 3900 automated DNA synthesizer (Applied Biosystems, Foster City, CA) was used with conventional phosphoramidite methods with minor changes to the coupling conditions for modified phosphoramidites. Modified phosphoramidites were used in 0.1 M solutions using acetonitrile with 0-40% dichloromethane and 0-20% sulfolane as the solvent. Solid support was an ABI style fritted column packed with controlled pore glass (CPG, LGC Biosearch Technologies, Petaluma CA) loaded with 3′-DMT-dT succinate with 1000 Å pore size. All syntheses were performed at the 50 nmole scale and the 5′ end of each sequence was modified with a photocleavable biotin/d-spacer followed by incorporation of TCOdU using phosphoramidite synthesized using the methods described in Example 2. Deprotection was accomplished by treatment with methylamine gas at 45° C. for 2 hours, the product mixtures were filtered, and the crude product mixture was purified preparatively on a Waters 2767 HPLC with a 2489 detector using a Hamilton PRP-H5 column. Purification was performed using a linear elution gradient that employed two buffers, (buffer A: 100 mM triethylammonium bicarbonate/5% acetonitrile, and buffer B: 100 mM triethylammonium bicarbonate/70% acetonitrile), with the gradient running at 80° C. from low buffer B content to high buffer B over the course of the elution. Product-containing fractions were combined and residual solvents evaporated in a Genevac HT-12 evaporator. Identity and percent full length product were determined using an Agilent 1290 Infinity with an Agilent 6130B single quadrupole mass spectrometry detector using an Acquity C18 column 1.7 μm 2.1×100 mm (Waters Corp, Milford, MA). See FIG. 2.

The resulting crude TCO-modified oligonucleotide residues were then redissolved in Water for Injection (WFI, HyPure WFI Quality Water, HyClone Laboratories, Logan, UT, or similar) to a 1-2 μM concentration. A commercially sourced sulfonated cyanine 3 tetrazine dye (Broad Pharm cat #BP-23321) was prepared in WFI water to 2.7 mM concentration. Each oligonucleotide mixture received an aliquot of the cyanine 3 tetrazine solution at a 2:1 ratio of tetrazine to oligonucleotide and the resulting mixture was mixed at room temperature for minimum 5 minutes, at which time analysis by LC/MS (Agilent 1290 Infinity, configured as above) confirmed that each reaction had reached a quantitative cycloaddition. See FIG. 2. The resulting products had four stereoisomers which may be observed on the resulting chromatogram as multiple peaks. See FIG. 2.

We claim:

1. A method of producing the compound: 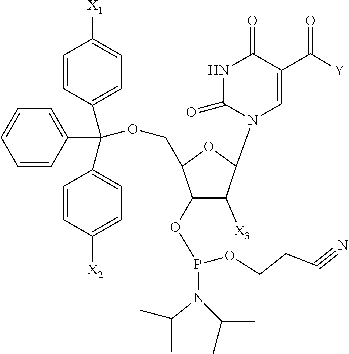
or a salt thereof,
wherein:
X₁ and X₂ are each independently selected from methoxy and hydrogen;
X₃ is selected from methoxy, fluoro, hydrogen, and tert-butyldimethylsilyloxy; and
Y is 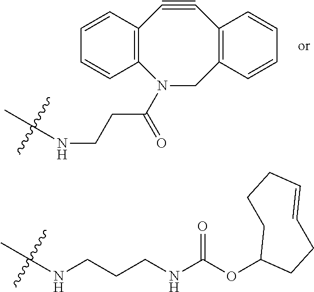 [SMILES: *NCCC(=O)N1Cc2ccccc2C#Cc2ccccc21 ; *NCCCNC(=O)OC1CC/C=C/CCC1]
the method comprising reacting the compound 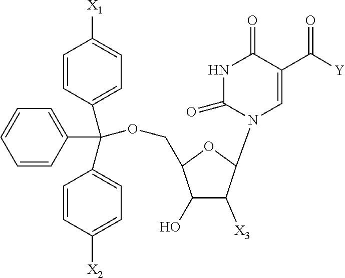
or a salt thereof, with 2-cyanoethyl N,N,N′,N′-tetraisopropylphosphorodiamidite and pyridine trifluoroacetic acid in dichloromethane; or 2-cyanoethyl N,N-diisopropyl chlorophosphoramidite and diisopropylethylamine in dichloromethane.

2. The method of claim 1, wherein:
Y is

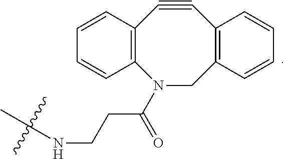 [SMILES: *NCCC(=O)N1Cc2ccccc2C#Cc2ccccc21]

3. The method of claim 2, wherein the compound: 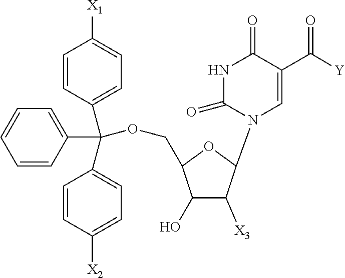
or a salt thereof, is prepared by reacting 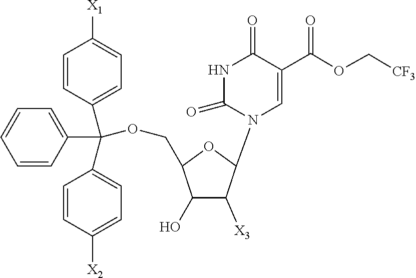
or a salt thereof, and 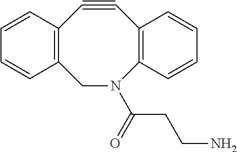 [SMILES: NCCC(=O)N1Cc2ccccc2C#Cc2ccccc21]
or a salt thereof, in the presence of triethylamine in acetonitrile.

4. The method of claim 1, wherein:
Y is

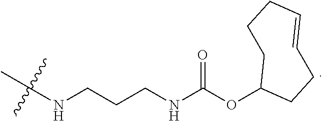 [SMILES: *NCCCNC(=O)OC1CC/C=C/CCC1]

5. The method of claim 4, wherein the compound: 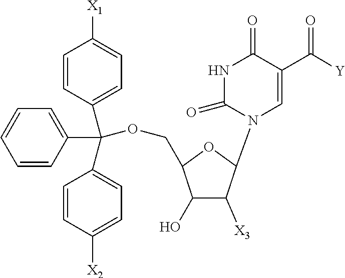
or a salt thereof, is prepared by reacting 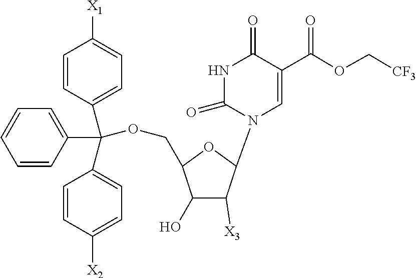
or a salt thereof, and 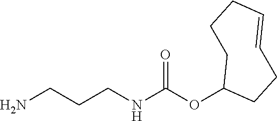 [SMILES: NCCCNC(=O)OC1CC/C=C/CCC1]
or a salt thereof, in the presence of triethylamine in acetonitrile.

6. The method of any one of claims 1-5, wherein X₁ and X₂ are both methoxy.

7. The method of any one of claims 1-6, wherein X₃ is hydrogen.

8. A compound of Formula (I): 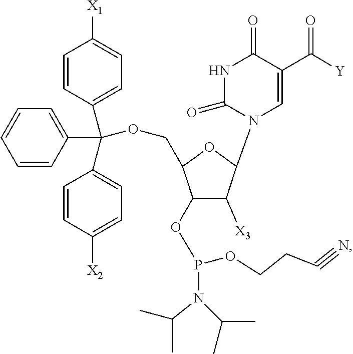
or a salt thereof,
wherein:
X₁ and X₂ are each independently selected from methoxy and hydrogen;
X₃ is selected from methoxy, fluoro, hydrogen, and tert-butyldimethylsilyloxy; and
Y is

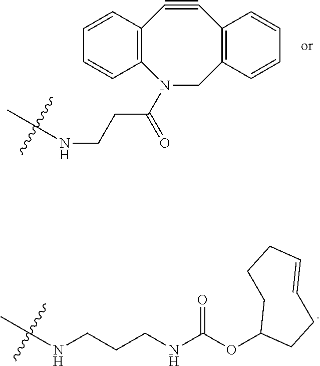 [SMILES: *NCCC(=O)N1Cc2ccccc2C#Cc2ccccc21 ; *NCCCNC(=O)OC1CC/C=C/CCC1]

9. A compound of Formula (II): 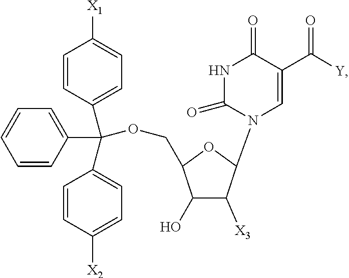
or a salt thereof,
wherein:
X₁ and X₂ are each independently selected from methoxy and hydrogen;
X₃ is selected from methoxy, fluoro, hydrogen, and tert-butyldimethylsilyloxy; and
Y is

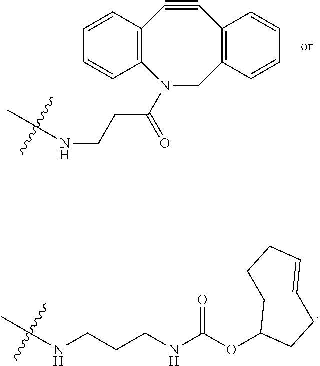 [SMILES: *NCCC(=O)N1Cc2ccccc2C#Cc2ccccc21 ; *NCCCNC(=O)OC1CC/C=C/CCC1]

10. The compound of claim 8 or claim 9, wherein X₁ and X₂ are both methoxy.

11. The compound of any one of claims 8-10, wherein X₃ is hydrogen.

12. An oligonucleotide comprising at least one click-labeled nucleotide, wherein at least one click-labeled nucleotide in the oligonucleotide has the structure: 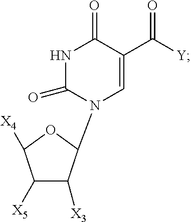
wherein:
X₃ is selected from a methoxy, fluoro, hydrogen, and tert-butyldimethylsilyloxy;
X₄ is selected from OH, —OR, —SR, and —Z—P(Z′)(Z″)O—R, wherein Z, Z′, and Z″ are each independently selected from O and S, and R is an adjacent nucleotide in the oligonucleotide;
X₅ is selected from —O-ss, —OR, —SR, and —Z—P(Z′)(Z″)O-T, wherein ss is a solid support, Z, Z′, and Z″ are each independently selected from O and S, and T is an adjacent nucleotide in the oligonucleotide; and
Y is

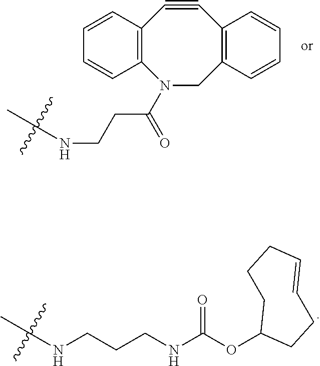 [SMILES: *NCCC(=O)N1Cc2ccccc2C#Cc2ccccc21 ; *NCCCNC(=O)OC1CC/C=C/CCC1]

13. An oligonucleotide comprising at least one 5-position modified nucleotide, wherein at least one modified nucleotide in the oligonucleotide has the structure: 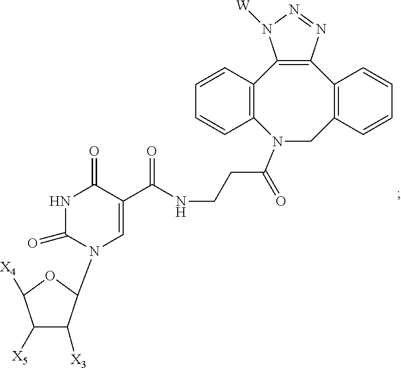
wherein:
X₃ is selected from a methoxy, fluoro, hydrogen, and tert-butyldimethylsilyloxy;
X₄ is selected from OH, —OR′, —SR′, and —Z—P(Z′)(Z″)O—R″, wherein Z, Z′, and Z″ are each independently selected from O and S, R′ is H or a cap, and R″ is H, a cap, or an adjacent nucleotide;
X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide; and
W is a payload moiety.

14. An oligonucleotide comprising at least one 5-position modified nucleotide, wherein at least one modified nucleotide in the oligonucleotide has the structure: 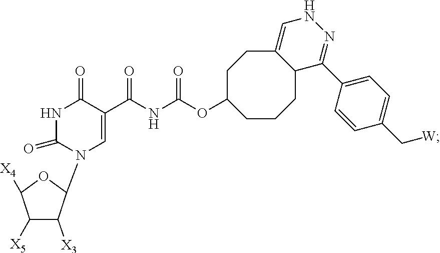
wherein:
X₃ is selected from a methoxy, fluoro, hydrogen, and tert-butyldimethylsilyloxy;
X₄ is selected from OH, —OR′, —SR′, and —Z—P(Z′)(Z″)O—R″, wherein Z, Z′, and Z″ are each independently selected from O and S, R′ is H or a cap, and R″ is H, a cap, or an adjacent nucleotide;
X₅ is selected from —O-ss, —OR′, —SR′, and —Z—P(Z′)(Z″)O—R″, wherein ss is a solid support, Z, Z′, and Z″ are each independently selected from O and S, R′ is H or a cap, and R″ is H, a cap, or an adjacent nucleotide; and
W is a payload moiety.

15. A method of producing an oligonucleotide comprising at least one 5-position modified nucleoside, comprising synthesizing an oligonucleotide comprising at least one nucleotide having the structure: 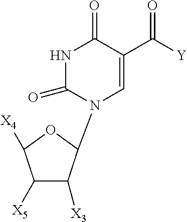
wherein:
X₃ is selected from a methoxy, fluoro, hydrogen, and tert-butyldimethylsilyloxy;
X₄ is selected from OH, —OR, —SR, and —Z—P(Z′)(Z″)O—R, wherein Z, Z′, and Z″ are each independently selected from O and S, and R is selected from H, a 5′ cap, and an adjacent nucleotide;
X₅ is selected from —O-ss, —OR, —SR, and —Z—P(Z′)(Z″)O—R, wherein ss is a solid support, Z, Z′, and Z″ are each independently selected from O and S, and R is selected from H and an adjacent nucleotide; and
Y is 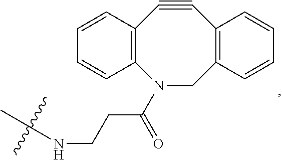 [SMILES: *NCCC(=O)N1Cc2ccccc2C#Cc2ccccc21]
and reacting the oligonucleotide with a reagent comprising a payload moiety and an azide moiety.

16. A method of producing an oligonucleotide comprising at least one 5-position modified nucleoside, comprising synthesizing an oligonucleotide comprising at least one nucleotide having the structure: 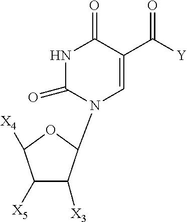
wherein:
X₃ is selected from a methoxy, fluoro, hydrogen, and tert-butyldimethylsilyloxy;
X₄ is selected from OH, —OR, —SR, and —Z—P(Z′)(Z″)O—R, wherein Z, Z′, and Z″ are each independently selected from O and S, and R is selected from H, a 5′ cap, and an adjacent nucleotide;
X₅ is selected from —O-ss, —OR, —SR, and —Z—P(Z′)(Z″)O—R, wherein ss is a solid support, Z, Z′, and Z″ are each independently selected from O and S, and R is selected from H and an adjacent nucleotide; and
Y is 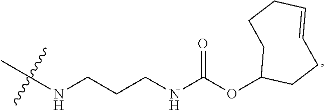 [SMILES: *NCCCNC(=O)OC1CC/C=C/CCC1]
and reacting the oligonucleotide with a reagent comprising a payload moiety and a tetrazine moiety.
